(19) United States
(12) Patent Application Publication
(10) Pub. No.: US 2019/0071406 A1
(43) Pub. Date: Mar. 7, 2019

(54) METHOD FOR PRODUCING ISOCYANURIC ACID DERIVATIVE HAVING TWO HYDROCARBON GROUPS

(71) Applicant: NISSAN CHEMICAL CORPORATION, Tokyo (JP)

(72) Inventors: Yuichi GOTO, Toyama-shi (JP); Gun SON, Toyama-shi (JP)

(73) Assignee: NISSAN CHEMICAL CORPORATION, Tokyo (JP)

(21) Appl. No.: 16/084,500

(22) PCT Filed: Feb. 27, 2017
(86) PCT No.: PCT/JP2017/007437

§ 371 Date: Sep. 12, 2018

(30) Foreign Application Priority Data
Mar. 16, 2016 (JP) 2016-052379

(51) Int. Cl. C07D 251/34

(52) U.S. Cl. CPC C07D 251/34

(57) ABSTRACT

A method for producing an isocyanuric acid derivative having two hydrocarbon groups including: a first step of obtaining a compound of Formula (1); a second step of obtaining a compound of Formula (2) from the compound of Formula (1); a third step of obtaining a compound of Formula (3) from the compound of Formula (2); and a fourth step of obtaining a compound of Formula (4) from the compound of Formula (3), wherein Bn is a benzyl group, and two Rs are each a C₁₋₁₀ hydrocarbon group, wherein all the steps are carried out at a temperature not exceeding 100° C.

### TECHNICAL FIELD

The present invention relates to a novel method for producing an isocyanuric acid derivative having two hydrocarbon groups as a substituent to be bonded to a nitrogen atom.

### BACKGROUND ART

An isocyanuric acid derivative and a method for synthesizing the same have been conventionally known. For example, Patent Document 1 describes a method for synthesizing 1,3-dimethyl-5-(2-hydroxyethyl) isocyanurate that is a specific example of an isocyanurate compound of the following Formula (I). A method for synthesizing 1,3-dimethyl isocyanurate used as a starting material for 1,3-dimethyl-5-(2-hydroxyethyl) isocyanurate is disclosed in pp. 397 to 398 of Non-Patent Document 1. Non-Patent Document 2 introduces a research result in which among 2,4,6-tris(benzyloxy)-1,3,5-triazine, 4,6-bis(benzyloxy)-1,3,5-triazine-2,4(1H,3H)-dione, and 6-(benzyloxy)-1,3,5-triazine-2,4(1H,3H)-dione, 6-(benzyloxy)-1,3,5-triazine-2,4(1H,3H)-dione is a benzylating reagent exhibiting the most excellent reactivity. Non-Patent Document 3 describes N-methylation using tetrabutylammonium isocyanurate. In the N-methylation, all mono-, di-, and tri-methyl isocyanurates are produced. Therefore, in a method described in Non-Patent Document 3, a mixture of the three types of N-methylated isocyanurates is always produced nonselectively.

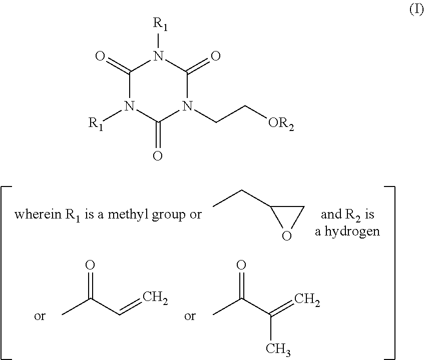

An isocyanuric acid derivative is used in various applications. For example, the isocyanurate compound described in Patent Document 1 is expected to be used as a raw material for a photocurable resin. Patent Document 2 describes a composition for forming an anti-reflective coating for use in a lithographic process containing an isocyanuric acid derivative. Patent Document 3 describes an adhesive composition containing a polymer obtained by polymerizing an isocyanuric acid derivative and a monomer other than the derivative.

### PRIOR ART DOCUMENTS

### Non-Patent Document

Non-Patent Document 1: Edwin M. Smolin; Lorence Rapoport. “Isocyanuric acid and derivatives”. The chemistry of heterocyclic compounds. s-Triazines and derivatives., INTERSCIENCE PUBLISHERS, INC., pp. 389 to 422(1959)
Non-Patent Document 2: Journal of Organic Chemistry, 80, pp. 11200 to 11205(2015)
Non-Patent Document 3: Tetrahedron Letters, 44, pp. 4399 to 4402(2003)

### PATENT DOCUMENTS

Patent Document 1: Japanese Patent Application Publication No. 2013-49657 (JP 2013-49657 A)
Patent Document 2: International publication WO2002/086624
Patent Document 3: International publication WO2013/035787

### SUMMARY OF THE INVENTION

### Problem to be Solved by the Invention

In a conventionally known method for producing a dialkylisocyanuric acid having two alkyl groups of isocyanuric acid derivatives, heating at a high temperature of 150° C. or higher for an extended period of time is required, and the selectivity of a reaction product is low. Therefore, this method is not industrially useful. Further, the method has a step in which safety is concerned as a problem.

### Means for Solving the Problems

The inventor of the present invention has found a method in which an isocyanuric acid derivative having two hydrocarbon groups is finally produced through steps of obtaining a first intermediate from cyanuric chloride (also referred to as 2,4,6-trichloro-1,3,5-triazine) that is relatively low cost and easily available, as a starting material, obtaining a second intermediate, and obtaining a third intermediate. That is, an aspect of the present invention is a method for producing an isocyanuric acid derivative having two hydrocarbon groups comprising a first step of obtaining a compound of the following Formula (1), a second step of obtaining a compound of the following Formula (2) from the compound of Formula (1), a third step of obtaining a compound of the following Formula (3) from the compound of Formula (2), and a fourth step of obtaining a compound of the following Formula (4) from the compound of Formula (3):

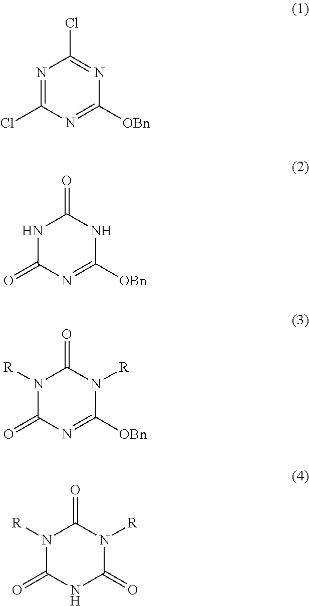

(wherein Bn is a benzyl group, and two Rs are each a C₁₋₁₀ hydrocarbon group), wherein all the steps are carried out at a temperature not exceeding 100° C.

For example, the C₁₋₁₀ hydrocarbon group is an alkyl group.

The first step is a process in which cyanuric chloride is reacted with benzyl alcohol in the presence of a base selected from the group consisting of a tertiary amine, an alkali metal carbonate, and an alkali metal hydrogen carbonate to obtain a reaction product, and the reaction product is washed with a solvent containing at least one selected from the group consisting of an aliphatic hydrocarbon solvent and an alcohol as a main component to obtain the compound of Formula (1) without purification through column chromatography. The benzyl alcohol and the base can be each used in an amount of 1.0 molar equivalent to 1.9 molar equivalents relative to 1.0 molar equivalent of the cyanuric chloride.

The base used in the first step may be a tertiary amine. Examples of the tertiary amine include diisopropylethylamine. As the base, an alkali metal carbonate or an alkali metal hydrogen carbonate may be used instead of the tertiary amine. Examples of the alkali metal carbonate include sodium carbonate. Examples of the alkali metal hydrogen carbonate include sodium hydrogen carbonate.

The main component of the solvent used for washing the reaction product in the first step may be an aliphatic hydrocarbon solvent. Examples of the aliphatic hydrocarbon solvent include heptane. The main component of the solvent may be an alcohol. Examples of the alcohol include ethanol. Only one type of the solvents may be selected for use, or two or more types of the solvents may be mixed at any ratio for use. Herein, the main component of the solvent is a component that is contained in the solvent in an amount of more than 50% by mass and 100% by mass or less, and preferably 80% by mass or more. The solvent may further contain a subcomponent. The subcomponent may be an ester. Examples of the ester include ethyl acetate.

The second step is a process in which a reaction product obtained by reacting the compound of Formula (1) in a solution containing N-methylmorpholine and an acetic acid salt is washed with water to obtain the compound of Formula (2).

Examples of the acetic acid salt used in the second step include sodium acetate.

Examples of water used for washing the reaction product in the second step include ultrapure water.

The third step is a process in which the compound of Formula (2) is reacted with any one of compounds of the following Formulae (5) to (8) having a C₁₋₁₀ hydrocarbon group in the presence of an alkali metal carbonate to obtain a reaction product, and to the reaction product, an aromatic hydrocarbon solvent and water are added, resulting in separation, to obtain the compound of Formula (3).

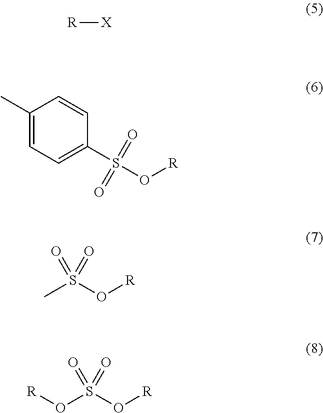

(wherein R is a C₁₋₁₀ hydrocarbon group, and X is a halogen atom.)

The one of the compounds of Formulae (5) to (8) used in the third step is, for example, an alkylating agent selected from the group consisting of halogenated alkyl, alkyl tosylate, alkyl mesylate, and dialkyl sulfate.

The alkylating agent may be halogenated alkyl or dialkyl sulfate. Examples of the halogenated alkyl include methyl iodide, ethyl bromide, and propyl bromide. Examples of the dialkyl sulfate include dimethyl sulfate. As the alkylating agent, alkyl tosylate or alkyl mesylate may be used instead of halogenated alkyl and dialkyl sulfate. Examples of the alkyl tosylate include methyl p-toluenesulfonate and ethyl p-toluenesulfonate. Examples of the alkyl mesylate include ethyl methanesulfonate.

Examples of the alkali metal carbonate used in the third step include potassium carbonate and cesium carbonate.

Examples of the aromatic hydrocarbon solvent used for separation of the reaction product in the third step include toluene. As water used with the aromatic hydrocarbon solvent, water used in the second step may be used.

The fourth step is a process in which the compound of Formula (3) is reacted with an alcohol compound in the presence of trifluoromethanesulfonic acid to obtain a reaction product, and the reaction product is washed with at least one solvent selected from the group consisting of an ester, a halogen-based solvent, and an alcohol to obtain the compound of Formula (4).

Examples of the alcohol compound used in the fourth step include methanol.

The at least one solvent used for washing the reaction product in the fourth step may be an ester. Examples of the ester include ethyl acetate. The at least one solvent may also be a halogen-based solvent or an alcohol. Examples of the halogen-based solvent include chloroform. Examples of the alcohol include ethanol.

The first to fourth steps are carried out at a temperature not exceeding 100° C. The temperature not exceeding 100° C. is 0° C. to 100° C., for example, 0° C. to 50° C.

### Effects of the Invention

A method for producing an isocyanuric acid derivative having two hydrocarbon groups according to the present invention does not include a step carried out at a temperature higher than 100° C. among all the steps, and does not require purification through column chromatography during purification of a reaction product. Therefore, this method is industrially useful. According to the present invention, the isocyanuric acid derivative having two hydrocarbon groups that is a target can be obtained at a high purity as compared with a conventional method.

### BRIEF DESCRIPTION OF THE DRAWINGS

FIG. 1 is a chromatogram illustrating a result of measurement of a compound of Example 1 obtained in a first step by high performance liquid chromatography.

FIG. 2 is a chromatogram illustrating a result of measurement of a compound of Example 4 obtained in a second step by high performance liquid chromatography.

FIG. 3 is a chromatogram illustrating a result of measurement of a compound of Example 5 obtained in a third step by high performance liquid chromatography.

FIG. 4 is a chromatogram illustrating a result of measurement of a compound of Example 6 obtained in a fourth step by high performance liquid chromatography.

### MODES FOR CARRYING OUT THE INVENTION

The method for producing an isocyanuric acid derivative having two hydrocarbon groups according to the present invention includes the first to fourth steps. Herewith, the compound of Formula (4) in which two hydrocarbon groups are introduced as a substituent bonded to a nitrogen atom of isocyanuric acid is obtained. In Formula (4), two Rs are each a C₁₋₁₀ hydrocarbon group introduced. The hydrocarbon group may be a linear, branched, or cyclic hydrocarbon group, and have at least one double bond or triple bond. When the hydrocarbon group is an alkyl group, examples of the alkyl group include methyl group, ethyl group, n-propyl group, isopropyl group, n-butyl group, isobutyl group, n-pentyl group, n-hexyl group, n-heptyl group, n-octyl group, n-pentyl group, n-nonyl group, n-decyl group, cyclohexylmethyl group, and cyclopentylmethyl group. Examples of the hydrocarbon group other than the alkyl group include benzyl group, allyl group, and propargyl group.

In the first step of the method of the present invention, cyanuric chloride is reacted with benzyl alcohol in the presence of a base such as a tertiary amine. In order to preferentially obtain the compound of Formula (1) in which only one chlorine atom of three chlorine atoms of cyanuric chloride is substituted with a benzyloxy group by this reaction, it is preferable that benzyl alcohol and the base such as a tertiary amine be each used in an amount of 1.0 molar equivalent to 1.9 molar equivalents relative to 1.0 molar equivalent of cyanuric chloride. When benzyl alcohol and the base such as a tertiary amine are each used in an amount of 2.0 molar equivalents relative to 1.0 molar equivalent of cyanuric chloride in the reaction, a compound in which two chlorine atoms of three chlorine atoms of cyanuric chloride are substituted with a benzyloxy group is produced as a by-product in substantially the same amount as that of the compound of Formula (1) that is a target, together with the compound of Formula (1). Therefore, the yield of the compound of Formula (1) is decreased.

Preferable examples of the tertiary amine used in the reaction include diisopropylethylamine, triethylamine, tributylamine, and 1,8-diazabicyclo[5.4.0]-7-undecene. When the reaction is carried out in a solution, it is preferable that a solvent be a halogen-based solvent. Examples of the halogen-based solvent include chloroform, dichloromethane, carbon tetrachloride, and dichloroethane.

Specifically, the reaction is started by adding a mixed solution of a benzyl alcohol, a base such as a tertiary amine and a halogen-based solvent dropwise to a mixed solution of cyanuric chloride and the halogen-based solvent. The temperature in the dropwise addition is preferably 0° C. to 5° C. When the addition dropwise is carried out at a temperature of higher than 5° C., a by-product is produced in addition to the compound of Formula (1) that is a target. Therefore, the purity and yield of the compound of Formula (1) are decreased. The reaction temperature after the dropwise addition is not particularly limited, and is usually 0° C. to 40° C., and preferably 20° C. to 30° C. The reaction time is usually 1 hour to 5 hours, and preferably 1 hour to 2 hours.

The reaction product obtained by the aforementioned reaction is separated, resulting in concentration. Subsequently, the reaction product is washed with a solvent containing at least one selected from the group consisting of an aliphatic hydrocarbon solvent and an alcohol as a main component, to obtain the compound of Formula (1). When the main component of the solvent used for the washing is the aliphatic hydrocarbon solvent, preferable examples thereof include heptane, hexane, octane, cyclopentane, cyclohexane, and cyclooctane. When the main component is the alcohol, preferable examples thereof include ethanol, methanol, isopropanol, n-propanol, sec-butyl alcohol, tert-butyl alcohol, n-butanol, and cyclohexanol. The amount of the solvent used in the washing is preferably 0.5 to 3.0 times the weight of the compound of Formula (1), and more preferably the same as the weight of the compound of Formula (1). The temperature in the washing is not particularly limited, and is usually 0° C. to 40° C., and preferably 20° C. to 30° C. The washing time is usually 10 minutes to 1 hour, and preferably 10 minutes to 30 minutes.

In the third step of the method of the present invention, the compound of Formula (2) is reacted with any one of compounds of Formulae (5) to (8) in the presence of an alkali metal carbonate. The amount of the one of the compounds of Formulae (5) to (8) to be used is preferably 2 molar equivalents to 3 molar equivalents, and more preferably 2.5 molar equivalents, relative to 1 molar equivalent of the compound of Formula (2). The amount of the alkali metal carbonate is preferably 2 molar equivalents to 3 molar equivalents, and more preferably 2.5 molar equivalents, relative to 1 molar equivalent of the compound of Formula (2). The temperature in the reaction is not particularly limited, and is usually 0° C. to 40° C., and preferably 20° C. to 30° C. The reaction time is usually 1 hour to 5 hours, and preferably 1 hour to 2 hours. When the reaction is carried out in a solution, it is preferable that a solvent be an aprotic polar solvent. Examples of the aprotic polar solvent include dimethyl sulfoxide, N-methylpyrrolidone, dimethylacetamide, and dimethylformamide.

When the one of the compounds of Formulae (5) to (8) used in the third step of the method of the present invention is selected, the type of hydrocarbon group to be introduced as a substituent bonded to a nitrogen atom of isocyanuric acid is determined. For example, as any one of the compounds of Formulae (5) to (8), a methyl group is introduced by using an alkylating agent having a methyl group, an ethyl group is introduced by using an alkylating agent having an ethyl group, a benzyl group is introduced by using a compound having a benzyl group (benzylating agent), an allyl group is introduced by using a compound having an allyl group (allylating agent), or a propargyl group is introduced by using a compound having a propargyl group. The one of the compounds of Formulae (5) to (8) is not limited to the aforementioned examples as long as it is a compound having a C₁₋₁₀ hydrocarbon group.

To a reaction product obtained by the aforementioned reaction, an aromatic hydrocarbon solvent and water are added, resulting in separation. As a result, the compound of Formula (3) is obtained. In order to remove the aprotic polar solvent used in the reaction by this separation, it is preferable that the aromatic hydrocarbon solvent and water be each used in an amount of 10 times the weight of the compound of Formula (3). The washing is preferably performed two times. When the washing is performed one time, the aprotic polar solvent cannot be removed. When washing is performed three times, the yield of the compound of Formula (3) is decreased. Preferable examples of the aromatic hydrocarbon solvent include toluene, benzene, xylene, mesitylene, chlorobenzene, dichlorobenzene, nitrobenzene, and tetralin. The temperature in the separation is not particularly limited, and is usually 0° C. to 40° C., and preferably 20° C. to 30° C.

In the fourth step of the method of the present invention, the compound of Formula (3) is reacted with an alcohol compound in the presence of trifluoromethanesulfonic acid. Examples of the alcohol compound include methanol, ethanol, isopropanol, n-propanol, sec-butyl alcohol, tert-butyl alcohol, n-butanol, cyclohexanol, and phenol. The amount of the alcohol compound used is preferably 1 molar equivalent to 1.5 molar equivalents, and more preferably 1.2 molar equivalents, relative to 1 molar equivalent of the compound of Formula (3). The amount of the trifluoromethanesulfonic acid used is preferably 0.01 molar equivalents to 2 molar equivalents, and more preferably 0.1 molar equivalents, relative to 1 molar equivalent of the compound of Formula (3). The temperature in the reaction is not particularly limited, and is usually 0° C. to 40° C., and preferably 20° C. to 30° C. The reaction time is usually 1 hour to 5 hours, and preferably 1 hour to 2 hours. When the reaction is carried out in a solution, it is preferable that a solvent be an ether. Examples of the ether include diethyl ether, diisopropyl ether, methyl tert-butyl ether, cyclopentyl methyl ether, tetrahydrofuran, and dioxane.

To a reaction product obtained by the aforementioned reaction, an organic base is added. Concentration is carried out. The reaction product is washed with at least one solvent selected from the group consisting of an ester, a halogen-based solvent, and an alcohol. As a result, the compound of Formula (4) is obtained. Examples of the organic base include pyridine, 4-dimethylaminopyridine, triethylamine, tributylamine, N,N-dimethylaniline, and 1,8-diazabicyclo[5.4.0]-7-undecene. The amount of the organic base used is preferably 0.1 molar equivalents to 1.0 molar equivalent, and more preferably 0.5 molar equivalents, relative to 1 molar equivalent of the compound of Formula (3). When the solvent used in the washing is the ester, preferable examples thereof include ethyl acetate, methyl acetate, butyl acetate, and methyl propionate. When the solvent is the halogen-based solvent, preferable examples thereof include chloroform, dichloromethane, carbon tetrachloride, and dichloroethane. When the solvent is the alcohol, preferable examples thereof include ethanol, methanol, isopropanol, n-propanol, sec-butyl alcohol, tert-butyl alcohol, n-butanol, and cyclohexanol. The amount of the solvent used in the washing is preferably 2.0 to 5.0 times, and more preferably 3.0 times the weight of the compound of Formula (3). The temperature in the washing is not particularly limited, and is usually 0° C. to 40° C., and preferably 20° C. to 30° C. The washing time is usually 10 minutes to 1 hour, and preferably 10 minutes to 30 minutes.

### EXAMPLES

Hereinafter, the method for producing an isocyanuric acid derivative having two hydrocarbon groups according to the present invention will be described with reference to specific examples. However, the present invention is not limited to the specific examples described below.

[HPLC Analysis Condition 1]

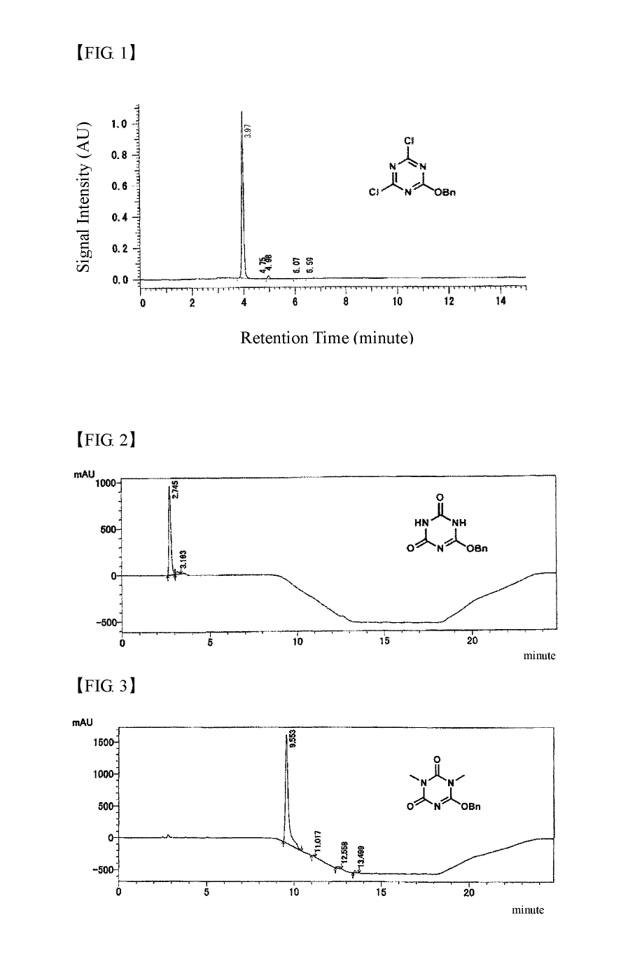

Purities shown in examples described below are results of measurement by high performance liquid chromatography (hereinafter abbreviated as HPLC). A measurement condition and the like are as follows. The purities are each obtained by dividing a peak of each component as shown in FIG. 1, and calculating the area of each peak of the respective components in percentage.

Apparatus: L2000 series manufactured by Hitachi High-Technologies Corporation
Column: XBridge (registered trademark) BEH C18 Column, 130 Å, 5 μm, 4.6 mm×250 mm (manufactured by Nihon Waters K.K.)
Eluent: acetonitrile/0.2% ammonium acetate aqueous solution=8/2 (v/v)
Flow rate: 1.0 mL/min

### Detector: UV (254 nm)

Column temperature: 40° C.
Analysis time: 15 minutes
Injection volume: 2.0 μL
Diluted solvent: acetonitrile

[HPLC Analysis Condition 2]

The purities shown in examples described below are results of measurement by HPLC. A measurement condition and the like are as follows. The purities are each obtained by dividing a peak of each component as shown in FIGS. 2, 3, and 4, and calculating the area of each peak in percentage.

Apparatus: LC-2010A manufactured by Shimadzu Corporation
Column: XBridge (registered trademark) BEH C18 Column, 130 Å, 5 μm, 4.6 mm×250 mm (manufactured by Nihon Waters K.K.)
Eluent: acetonitrile/0.2% ammonium acetate aqueous solution=3/7 (v/v) (0 minute to 5 minutes), the composite ratio was changed from 3/7 (v/v) to 8/2 (v/v) (5 minutes to 10 minutes), and 8/2 (v/v) (10 minutes to 15 minutes)
Flow rate: 1.0 mL/min

### Detector: UV (210 nm)

column temperature: 40° C.
Analysis time: 25 minutes
Injection volume: 1.0 μL
Diluted solvent: acetonitrile/water=1/1 (w/w)

[First Step]

### Example 1

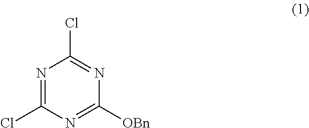 [SMILES: Clc1nc(Cl)nc(OCc2ccccc2)n1]

15.00 g of cyanuric chloride (available from Tokyo Chemical Industry Co., Ltd.) and 150.00 g of dichloromethane were mixed, and the mixture was cooled to 0° C. with stirring. To the mixture, a mixed solution of 8.80 g of benzyl alcohol (available from Kanto Chemical Co., Inc.), 11.56 g of diisopropylethylamine, and 60.00 g of dichloromethane was added dropwise. After completion of dropwise addition, the temperature was increased to 25° C. and a reaction solution was stirred for 1.5 hours. To the reaction solution, 150.00 g of 1 M HCl was added, resulting in separation. To an organic phase, 150.00 g of saturated saline solution was added, resulting in separation. This separation operation was repeated twice. From the organic phase, a solvent was distilled off under reduced pressure, and a residue was then dried at 40° C. under reduced pressure. To the residue, 15.0 g of heptane was added, and the mixture was stirred at 25° C. for 10 minutes. After stirring, the mixture was filtered, and a cake was washed with 15.0 g of heptane twice. Herein, the cake is a solid material that remains after a liquid is separated by filtration of a solid-liquid mixture such as a slurry. The resulting crystal was dried at 40° C. under reduced pressure, to obtain 17.31 g of triazine compound of Formula (1) described above as a pale yellow solid (yield: 83.1%). The obtained compound was subjected to measurement under the HPLC analysis condition 1. The purity thereof was 97.8%. A chromatogram obtained by the measurement is shown in FIG. 1.

### Example 2

16.43 g of triazine compound of Formula (1) as a pale yellow solid (yield: 78.9%) was obtained in the same manner as in Example 1 except that a washing solvent was changed from heptane to ethyl acetate/heptane (1/9(w/w/)). The obtained compound was subjected to measurement under the HPLC analysis condition 1. The purity thereof was 98.1%.

### Example 3

16.58 g of triazine compound of Formula (1) as a pale yellow solid (yield: 79.6%) was obtained in the same manner as in Example 1 except that a washing solvent was changed from heptane to ethanol. The obtained compound was subjected to measurement under the HPLC analysis condition 1. The purity thereof was 98.3%.

### Comparative Example 1

A residue was obtained in the same manner as in Example 1 except that a washing solvent was changed from heptane to toluene. After stirring, the residue was completely dissolved. As a result, a crystal was not obtained.

### Comparative Example 2

3.06 g of triazine compound of Formula (1) as a pale yellow solid (yield: 14.7%) was obtained in the same manner as in Example 1 except that a washing solvent was changed from heptane to ethyl acetate. The obtained compound was subjected to measurement under the HPLC analysis condition 1. The purity thereof was 98.6%.

### Comparative Example 3

5.00 g of cyanuric chloride (available from Tokyo Chemical Industry Co., Ltd.) and 50.00 g of dichloromethane were mixed, and the mixture was cooled to 0° C. with stirring. To the mixture, a mixed solution of 2.93 g of benzyl alcohol (available from Kanto Chemical Co., Inc.), 3.86 g of diisopropylethylamine, and 20.00 g of dichloromethane was added dropwise. After completion of dropwise addition, the temperature was increased to 25° C. and a reaction solution was stirred for 1 hour. To the reaction solution, 50.00 g of 1 M HCl was added, resulting in separation. To an organic phase, 50.00 g of saturated saline solution was added, resulting in separation. This separation operation was repeated twice. From the organic phase, a solvent was distilled off under reduced pressure, and a residue was then dried at 40° C. under reduced pressure. The residue was subjected to silica gel chromatography using chloroform/hexane (1/1 (w/w)) as a developing solvent. The obtained solution was concentrated at 40° C. and dried under reduced pressure, to obtain 5.8 g of triazine compound of Formula (1) as a white solid (yield: 84.7%). The obtained compound was subjected to measurement under the HPLC analysis condition 1. The purity thereof was 77.9%.

### Comparative Example 4

To 5.8 g of the triazine compound of Formula (1) obtained in Comparative Example 3, 5.0 g of hexane was added, and the mixture was stirred at 25° C. for 10 minutes. After stirring, the mixture was filtered, and a cake was washed with 5.0 g of hexane twice. The resulting crystal was dried at 40° C. under reduced pressure, to obtain 5.50 g of triazine compound of Formula (1) as a white solid (yield: 79.1%). The obtained compound was subjected to measurement under the HPLC analysis condition 1. The purity thereof was 97.4%.

TABLE 1

| | | Yield (%) | Purity (%) |
|---|---|---|---|
| Example 1 | Heptane | 83.1 | 97.8 |
| Example 2 | Ethyl | 78.9 | 98.1 |
| | acetate/heptane | | |
| | (1/9(w/w)) | | |
| Example 3 | Ethanol | 79.6 | 98.3 |

| | | |
|---|---|---|
| Comparative | Toluene | Dissolved |
| Example 1 | | |

| | | | |
|---|---|---|---|
| Comparative | Ethyl acetate | 14.7 | 98.6 |
| Example 2 | | | |
| Comparative | Column | 84.7 | 77.9 |
| Example 3 | chromatography | | |
| Comparative | Washing with | 79.1 | 97.4 |
| Example 4 | hexane after | | |
| | column | | |
| | chromatography | | |

[Second Step]

### Example 4

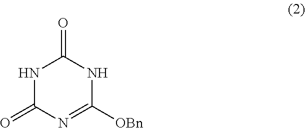 [SMILES: O=c1nc(OCc2ccccc2)[nH]c(=O)[nH]1]

3.95 g of N-methyl morpholine (available from Tokyo Chemical Industry Co., Ltd.), 81.30 g of sodium acetate (available from Kanto Chemical Co., Inc.), and 450.00 g of water were mixed, and the mixture was cooled to 0° C. To the mixture, a mixed solution of 50.00 g of the triazine compound of Formula (1) obtained in Example 1 and 450.00 g of tetrahydrofuran was added dropwise with stirring at a temperature not exceeding 10° C. After completion of dropwise addition, the temperature was increased to 25° C. and a reaction solution was stirred for 46 hours. To the reaction solution, 50.00 g of 1 M HCl was added, and a solvent was then removed under reduced pressure until the weight was 675.00 g. The deposited crystal was filtered, and a cake was washed with 50.00 g of water twice. Subsequently, a residue was dried at 40° C. under reduced pressure, to obtain a crystal. To the crystal, 150.00 g of ethanol was added, and the mixture was stirred at 25° C. for 10 minutes. After stirring, the mixture was filtered, and a cake was washed with 25.00 g of ethanol twice. A residue was dried at 40° C. under reduced pressure, to obtain 29.49 g of triazinedione compound of Formula (2) (yield: 68.9%). The obtained compound was subjected to measurement under the HPLC analysis condition 2. The purity thereof was 95.7%. A chromatogram obtained by the measurement is shown in FIG. 2.

[Third Step]

### Example 5

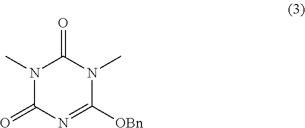 [SMILES: Cn1c(OCc2ccccc2)nc(=O)n(C)c1=O]

10.00 g of the triazinedione compound obtained in Example 4, 37.16 g of cesium carbonate (available from Tokyo Chemical Industry Co., Ltd.), and 100.00 g of dimethyl sulfoxide were mixed, and the mixture was stirred at 25° C. To the mixture, 16.19 g of iodomethane (available from Tokyo Chemical Industry Co., Ltd.) was added dropwise. After completion of dropwise addition, the mixture was stirred at 25° C. for 1 hour to obtain a reaction solution. To the reaction solution, 100.00 g of toluene was added, and the mixture was filtered. To the filtrate, 100.00 g of water was added, resulting in separation. This separation operation was repeated twice. From the organic phase, a solvent was distilled off under reduced pressure, and a residue was then dried at 40° C. under reduced pressure. As a result, 9.14 g of dimethyltriazinedione compound of Formula (3) described above was obtained (yield: 81.0%). The obtained compound was subjected to measurement under the HPLC analysis condition 2. The purity thereof was 99.3%. A chromatogram obtained by the measurement is shown in FIG. 3. This compound was subjected to ¹H NMR measurement (500 MHz, CDCl₃). The measurement result was δ 7.49-7.37 (m, 5H), 5.42 (s, 2H), 3.23 (s, 3H), 3.15 (s, 3H)

A NMR device used in ¹H NMR measurement was JNM-ECA500 manufactured by JEOL Ltd.

### Comparative Example 5

A reaction was carried out in the same manner as in Example 4 until a reaction solution was obtained. To the reaction solution, 100.00 g of diethyl ether was added, and the mixture was filtered. Subsequently, separation was carried out, and a dimethyl sulfoxide phase was removed. To a diethyl ether phase, 100.00 g of water was added, resulting in separation. This separation operation was repeated twice. From the organic phase, a solvent was distilled off under reduced pressure, and a residue was then dried at 40° C. under reduced pressure. As a result, 3.30 g of dimethyltriazinedione compound of Formula (3) was obtained (yield: 26.5%). The obtained compound was subjected to measurement under the HPLC analysis condition 2. The purity thereof was 98.8%.

### Comparative Example 6

A reaction was carried out in the same manner as in Example 4 until a reaction solution was obtained. 2.00 g of dimethyltriazinedione compound of Formula (3) was obtained (yield: 18.0%) in the same manner as in Example 4 except that cyclopentyl methyl ether was added to the reaction solution. The obtained compound was subjected to measurement under the HPLC analysis condition 2. The purity thereof was 94.0%.

### Comparative Example 7

A reaction was carried out in the same manner as in Example 4 until a reaction solution was obtained. 7.70 g of dimethyltriazinedione compound of Formula (3) was obtained (yield: 68.5%) in the same manner as in Example 4 except that ethyl acetate was added to the reaction solution. The obtained compound was subjected to measurement under the HPLC analysis condition 2. The purity thereof was 96.2%.

TABLE 2

| | | Yield (%) | Purity (%) |
|---|---|---|---|
| Example 5 | Toluene | 81.0 | 99.3 |
| Comparative | Diethyl ether | 26.5 | 98.8 |
| Example 5 | | | |
| Comparative | Cyclopentyl methyl | 18.0 | 94.0 |
| Example 6 | ether | | |
| Comparative | Ethyl acetate | 68.5 | 96.2 |
| Example 7 | | | |

[Fourth Step]

### Example 6

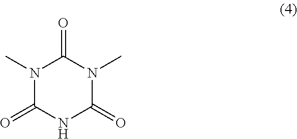

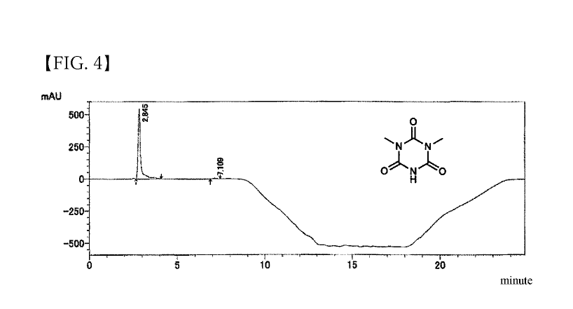

1.00 g of the dimethyltriazinedione compound obtained in Example 5, 10.00 g of 1,4-dioxane, and 0.16 g of methanol were mixed. To the mixture, 0.06 g of trifluoromethanesulfonic acid (available from Tokyo Chemical Industry Co., Ltd.) was added dropwise with stirring at 25° C. After completion of dropwise addition, the mixture was stirred at 25° C. for 2 hours to obtain a reaction solution. To the reaction solution, 0.20 g of triethylamine was added. A solvent was distilled off under reduced pressure, and a residue was then dried at 40° C. under reduced pressure. To the residue, 3.00 g of ethyl acetate was then added, and the mixture was stirred at 25° C. for 10 minutes. After stirring, the mixture was filtered, and a cake was washed with 1.00 g of ethyl acetate twice. The resulting crystal was dried at 40° C. under reduced pressure, to obtain 0.46 g of dimethylisocyanuric acid of Formula (4) as a white solid (yield: 71.9%). The obtained compound was subjected to measurement under the HPLC analysis condition 2. The purity thereof was 99.1%. A chromatogram obtained by the measurement is shown in FIG. 4. This compound was subjected to ¹H NMR measurement (500 MHz, DMSO-d₆). The measurement result was δ 11.64 (s, 1H), 3.10 (s, 6H)

A NMR device used in ¹H NMR measurement was JNM-ECA500 manufactured by JEOL Ltd.

### Example 7

0.48 g of dimethylisocyanuric acid of Formula (4) as a white solid (yield: 75.0%) was obtained in the same manner as in Example 5 except that a washing solvent was changed from ethyl acetate to chloroform. The obtained compound was subjected to measurement under the HPLC analysis condition 2. The purity thereof was 98.9%.

### Example 8

0.43 g of dimethylisocyanuric acid of Formula (4) as a white solid (yield: 66.5%) was obtained in the same manner as in Example 5 except that a washing solvent was changed from ethyl acetate to ethanol. The obtained compound was subjected to measurement under the HPLC analysis condition 2. The purity thereof was 99.1%.

### Comparative Example 8

0.58 g of dimethylisocyanuric acid of Formula (4) as a pale yellow solid (yield: 90.6%) was obtained in the same manner as in Example 5 except that a washing solvent was changed from ethyl acetate to toluene. The obtained compound was subjected to measurement under the HPLC analysis condition 2. The purity thereof was 96.2%.

### Comparative Example 9

0.69 g of dimethylisocyanuric acid of Formula (4) as a pale yellow solid (yield: >99.9%) was obtained in the same manner as in Example 5 except that a washing solvent was changed from ethyl acetate to heptane. The obtained compound was subjected to measurement under the HPLC analysis condition 2. The purity thereof was 89.1%.

### Comparative Example 10

0.64 g of dimethylisocyanuric acid of Formula described above as a pale yellow solid (yield: 99.4%) was obtained in the same manner as in Example 5 except that a washing solvent was changed from ethyl acetate to cyclopentyl methyl ether. The obtained compound was subjected to measurement under the HPLC analysis condition 2. The purity thereof was 92.0%.

TABLE 3

| | | Yield (%) | Purity (%) |
|---|---|---|---|
| Example 6 | Ethyl acetate | 71.9 | 99.1 |
| Example 7 | Chloroform | 75.0 | 98.9 |
| Example 8 | Ethanol | 66.5 | 99.1 |
| Comparative | Toluene | 90.6 | 96.2 |
| Example 8 | | | |
| Comparative | Heptane | >99.9 | 89.1 |
| Example 9 | | | |
| Comparative | Cyclopentyl methyl | 99.4 | 92.0 |
| Example 10 | ether | | |

### INDUSTRIAL APPLICABILITY

For example, an isocyanuric acid derivative having two hydrocarbon groups produced by the present invention can be applied to an anti-reflective coating-forming composition for lithography, a resist underlayer film-forming composition, a resist upper layer film-forming composition, a photocurable resin composition, a thermosetting resin composition, a flattened film-forming composition, an adhesives composition, and other compositions.

1. A method for producing an isocyanuric acid derivative having two hydrocarbon groups comprising:
a first step of obtaining a compound of Formula (1) described below;
a second step of obtaining a compound of Formula (2) described below from the compound of Formula (1);
a third step of obtaining a compound of Formula (3) described below from the compound of Formula (2); and
a fourth step of obtaining a compound of Formula (4) described below from the compound of Formula (3), 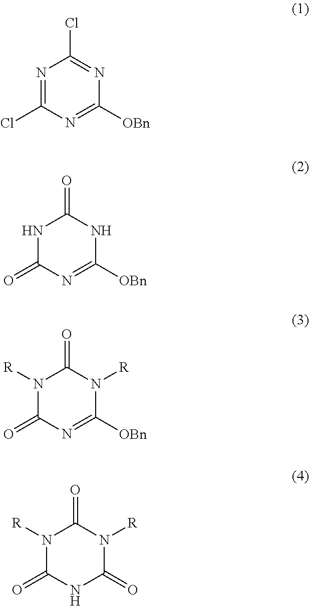
(wherein Bn is a benzyl group, and two Rs are each a C₁₋₁₀ hydrocarbon group),
wherein the first step is a process in which cyanuric chloride is reacted with benzyl alcohol in the presence of a base selected from the group consisting of a tertiary amine, an alkali metal carbonate, and an alkali metal hydrogen carbonate to obtain a reaction product, and the reaction product is washed with a solvent containing at least one selected from the group consisting of an aliphatic hydrocarbon solvent and an alcohol as a main component to obtain the compound of Formula (1) without purification through column chromatography,
the third step is a process in which the compound of Formula (2) is reacted with any one of compounds of Formulae (5) to (8) described below having a C₁₋₁₀ hydrocarbon group in the presence of an alkali metal carbonate to obtain a reaction product, and to the reaction product, an aromatic hydrocarbon solvent and water are added, resulting in separation, to obtain the compound of Formula (3), 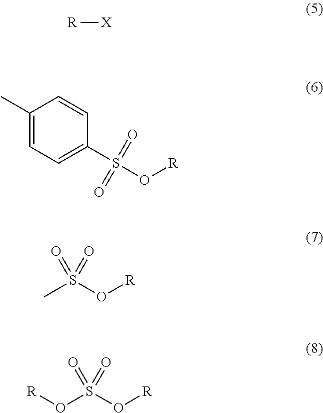
(wherein R is a C₁₋₁₀ hydrocarbon group, and X is a halogen atom),
the fourth step is a process in which the compound of Formula (3) is reacted with an alcohol compound in the presence of trifluoromethanesulfonic acid to obtain a reaction product, and the reaction product is washed with at least one solvent selected from the group consisting of an ester, a halogen-based solvent, and an alcohol to obtain the compound of Formula (4), and all the steps are carried out at a temperature not exceeding 100° C.

2. The method for producing an isocyanuric acid derivative having two hydrocarbon groups according to claim 1, wherein the C₁₋₁₀ hydrocarbon group is an alkyl group.

3. The method for producing an isocyanuric acid derivative having two hydrocarbon groups according to claim 1, wherein in the first step, the benzyl alcohol and the base are each used in an amount of 1.0 molar equivalent to 1.9 molar equivalents relative to 1.0 molar equivalent of the cyanuric chloride.

4. The method for producing an isocyanuric acid derivative having two hydrocarbon groups according to claim 1, wherein the base is a tertiary amine.

5. The method for producing an isocyanuric acid derivative having two hydrocarbon groups according to claim 1, wherein the at least one solvent used in the first step is selected from the group consisting of heptane and ethanol.

6. The method for producing an isocyanuric acid derivative having two hydrocarbon groups according to claim 1, wherein in the second step, a reaction product obtained by reacting the compound of Formula (1) in a solution containing N-methylmorpholine and an acetic acid salt is washed with water to obtain the compound of Formula (2).

7. The method for producing an isocyanuric acid derivative having two hydrocarbon groups according to claim 1, wherein the one of compounds of Formulae (5) to (8) is an alkylating agent selected from the group consisting of halogenated alkyl, alkyl tosylate, alkyl mesylate, and dialkyl sulfate.

8. The method for producing an isocyanuric acid derivative having two hydrocarbon groups according to claim 7, wherein the alkylating agent is methyl iodide, ethyl bromide, propyl bromide, dimethyl sulfate, methyl p-toluenesulfonate, ethyl p-toluenesulfonate, or ethyl methanesulfonate.

9. The method for producing an isocyanuric acid derivative having two hydrocarbon groups according to claim 1, wherein the alkali metal carbonate used in the third step is potassium carbonate or cesium carbonate.

10. The method for producing an isocyanuric acid derivative having two hydrocarbon groups according to claim 1, wherein the aromatic hydrocarbon solvent is toluene.

11. The method for producing an isocyanuric acid derivative having two hydrocarbon groups according to claim 1, wherein the at least one solvent used in the fourth step is selected from the group consisting of ethyl acetate, chloroform, and ethanol.

12. The method for producing an isocyanuric acid derivative having two hydrocarbon groups according to claim 1, wherein the temperature not exceeding 100° C. is 0° C. to 50° C.
